(19) United States
(12) Patent Application Publication
(10) Pub. No.: US 2017/0020717 A1
(43) Pub. Date: Jan. 26, 2017

(54) Condom with Side Portals

(71) Applicant: Hong Wang, Staten Island, NY (US)

(72) Inventor: Hong Wang, Staten Island, NY (US)

(21) Appl. No.: 15/273,117

(22) Filed: Sep. 22, 2016

(51) Int. Cl. A61F 6/04

(52) U.S. Cl. CPC A61F 6/04

(57) ABSTRACT

A condom of embodiments of the disclosed technology has a tip and base with cords connecting the two, creating portals where the urethra is exposed and fully capable of feeling abrasion with other objects. The base ring is oppositely disposed from the tip and an extreme other end of the condom. The base ring also extends circumferentially and in parallel to a largest circumference of the solid tip, when the cord extends perpendicularly to the largest circumference of the solid tip and the base ring. The largest circumference of the circle referred to herein, in embodiments of the disclosed technology, is a portion of the tip which is closest to the base ring.

### FIELD OF THE DISCLOSED TECHNOLOGY

The disclosed technology relates generally to condoms and, more specifically, to condoms with openings therein.

### BACKGROUND OF THE DISCLOSED TECHNOLOGY

A condom is used as a barrier between the penis and the vagina, in order to prevent sperm that leaves the urethra from entering into the vaginal cavity and thereby transferring disease or impregnating a woman. While that is all well and good, no one in their right mind wants to use a condom because it is also a torture device. The pleasurable sensation which comes along with inserting one's male member into a female counterpart is a fraction of what it would be, when using the condom. They don't tell this to kids in health class, so the obedient child, who does what he's led to believe is the right thing, finds out that listening to authority was a rather silly idea. After putting a condom on his member, he could slam in a car door and wouldn't feel a thing. Now what his health teacher said about pre-marital sex is out the window, and he next thing you know, he's not listening to what his health class teacher said about drugs either, and we have a meth addict all because of condoms.

Some have tried to compensate for this problem by covering only the tip of the urethra, such as in German patent publication DE202004013724.The problem with this is that the top is liable to slip off, and a false sense of protection is worse than no protection at all. Without the tip cover, one might not have engaged in the activity at all, and now that they have, they might very well have a problem large enough to make an atheist pray to his maker.

What is needed is a way of protecting ejaculated matter from entering a woman, while at the same time, keeping the pleasure of the act intact.

### SUMMARY OF THE DISCLOSED TECHNOLOGY

A condom of embodiments of the disclosed technology has a solid tip. “Solid” is defined as being impermeable to liquid. At least one cord, such as one, two or four, extends from the tip to a base ring. The base ring is oppositely disposed from the tip and an extreme other end of the condom. The base ring also extends circumferentially and in parallel to a largest circumference of the solid tip, when the cord extends perpendicular to the largest circumference of the solid tip and the base ring. A “circumference,” for purposes of this disclosure, refers to a mathematical circumference of a circle or a shape which appears to be, or is called, a circle by an ordinary observer. The largest circumference of the circle referred to herein, in embodiments of the disclosed technology, is a portion of the tip which is closest to the base ring.

When there are two cords, in embodiments, they are in parallel to each other and connect 180 degrees apart from each other to each of the base ring and the largest circumference of the solid tip. When there are four cords, in embodiments, they are in parallel to one another and connect 90 or 180 degrees apart from each other to each of the base ring and the largest circumference of the solid tip.

A plurality of lateral rings, in embodiment, crosses over each of the one, two, or four cords and is parallel to the base ring and the largest circumference of the solid tip. The lateral rings can be equi-spaced from each other and there can be 1, 2, 3, 4, 5, 6, 7, 8, 9, or 10 cords. Instead of lateral rings, there can be diamonds formed from diagonals which are like cords, except that they are at a 45 degree angle to the base ring and, if existing in the particular embodiment, the cord or cords. There can be one set of such described diagonals, or two. When there are two, each set is at 180 degrees to each other. Triangular portals are formed with a hypotenuse along a cord of the at least one cord, and each leg is along a diagonal of the plurality of diagonals, in some embodiments.

Further discussing the portals, a portal, for purposes of this disclosure, is defined as a space between solid material through which liquid or a solid can pass. It can also be a space where the urethra of a penis is exposed while wearing a condom of the disclosed technology. At least one portal extends from the tip to the base ring, except where interrupted by a cord. When one cord interrupts, there is one portal. When two cords interrupt, there are two portals. When four cords interrupt, there are four portals.

The tip can be configured to cover a tip of urethra, the base ring configured to cover a lower portion of the urethra, and an inner cavity can be defined by space between the base ring, the at least one cord, and the tip. The inner cavity thus can also be configured to be filled with a urethra. For purposes of this disclosure, “filled” is defined as at least 95% water impermeable material (e.g., a urethra with skin) within the space that is filled.

The condom can be procured from a single piece of plastic or polymer material. The material can be uninterrupted by any other, continuous without a break, and even formed all simultaneously, such as through an extrusion or molding process. One can use the condom by pulling the solid tip over a tip of the urethra, pulling the base ring over a lower part of a urethra, straightening the at least one cord until the base ring is parallel to the largest circumference of the tip, and engaging in an activity which causes ejaculation into the tip while the base ring remains substantially in place.

“Substantially” and “substantially shown,” for purposes of this specification, are defined as “at least 90%,” or as otherwise indicated. Any device may “comprise” or “consist of” the devices mentioned there-in, as limited by the claims.

It should be understood that the use of “and/or” is defined inclusively such that the term “a and/or b” should be read to include the sets: “a and b,” “a or b,” “a” “b”.

### BRIEF DESCRIPTION OF THE DRAWINGS

FIG. 1 shows a top perspective view of a portal condom, in an embodiment of the disclosed technology.

FIG. 2 shows a bottom perspective view of the portal condom of FIG. 1.

FIG. 3 shows a side elevation view of the portal condom of FIG. 1.

FIG. 4 shows a top plan view of the portal condom of FIG. 1.

FIG. 5 shows a bottom plan view of the portal condom of FIG. 1.

FIG. 6 shows a perspective view of the portal condom of FIG. 1, with a urethra inserted therein.

FIG. 7 shows an alternative embodiment of the portal condom with cross-connected rings, in an embodiment of the disclosed technology.

FIG. 8 shows a second alternative embodiment of the portal condom with diamond connected rings, in an embodiment of the disclosed technology.

FIG. 9 shows a third alternative embodiment of the portal condom with two cords.

FIG. 10 shows a third alternative embodiment of the portal condom with one cords.

### DETAILED DESCRIPTION OF EMBODIMENTS OF THE DISCLOSED TECHNOLOGY

A condom of embodiments of the disclosed technology has a tip and base, with cords connecting the two, creating portals where the urethra is exposed and fully capable of feeling abrasion with other objects. The base ring is oppositely disposed from the tip and an extreme other end of the condom. The base ring also extends circumferentially and in parallel to a largest circumference of the solid tip, when the cord extends perpendicular to the largest circumference of the solid tip and the base ring. The largest circumference of the circle referred to herein, in embodiments of the disclosed technology, is a portion of the tip which is closest to the base ring.

Embodiments of the disclosed technology will become clearer in view of the following discussion of the figures.

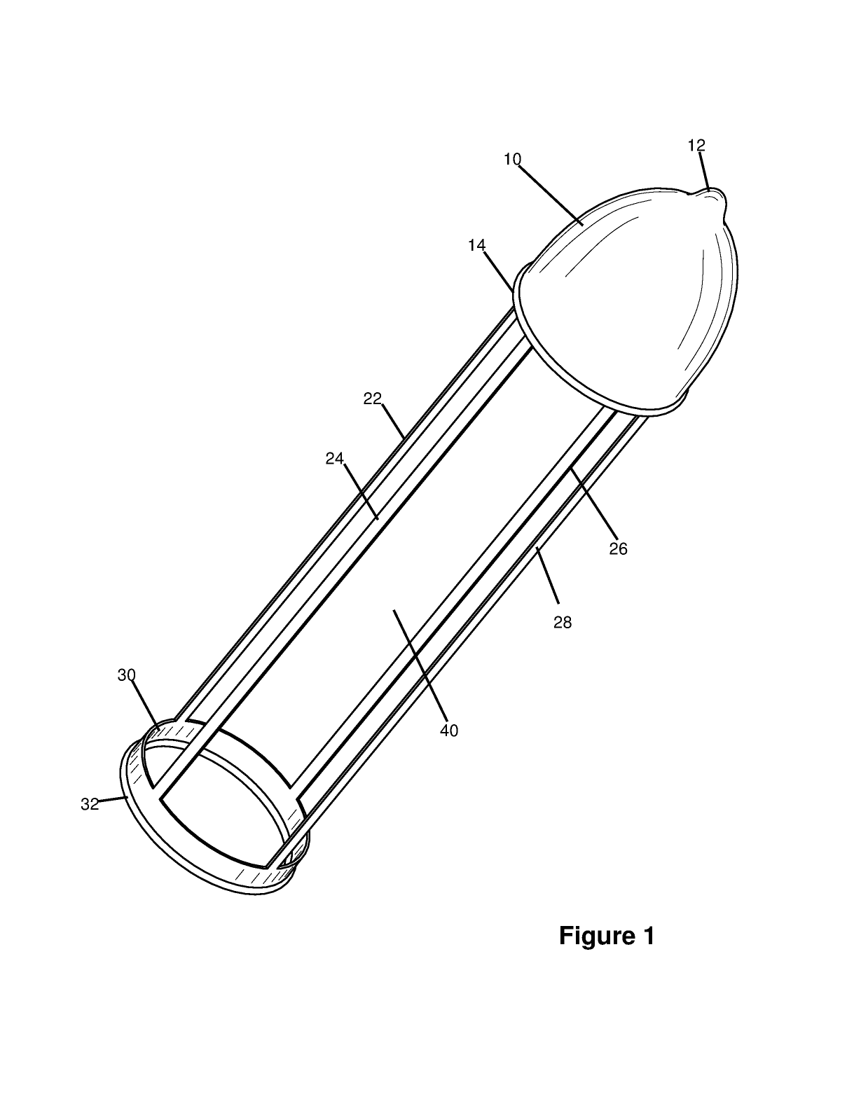

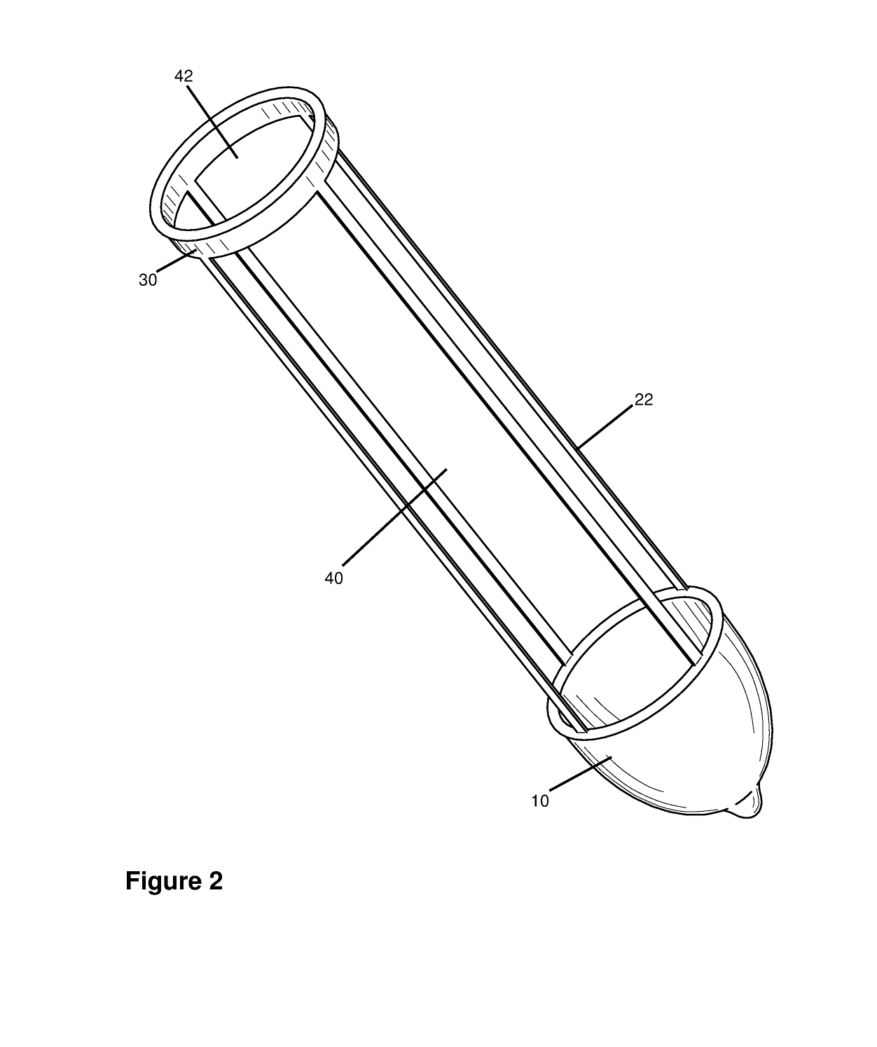

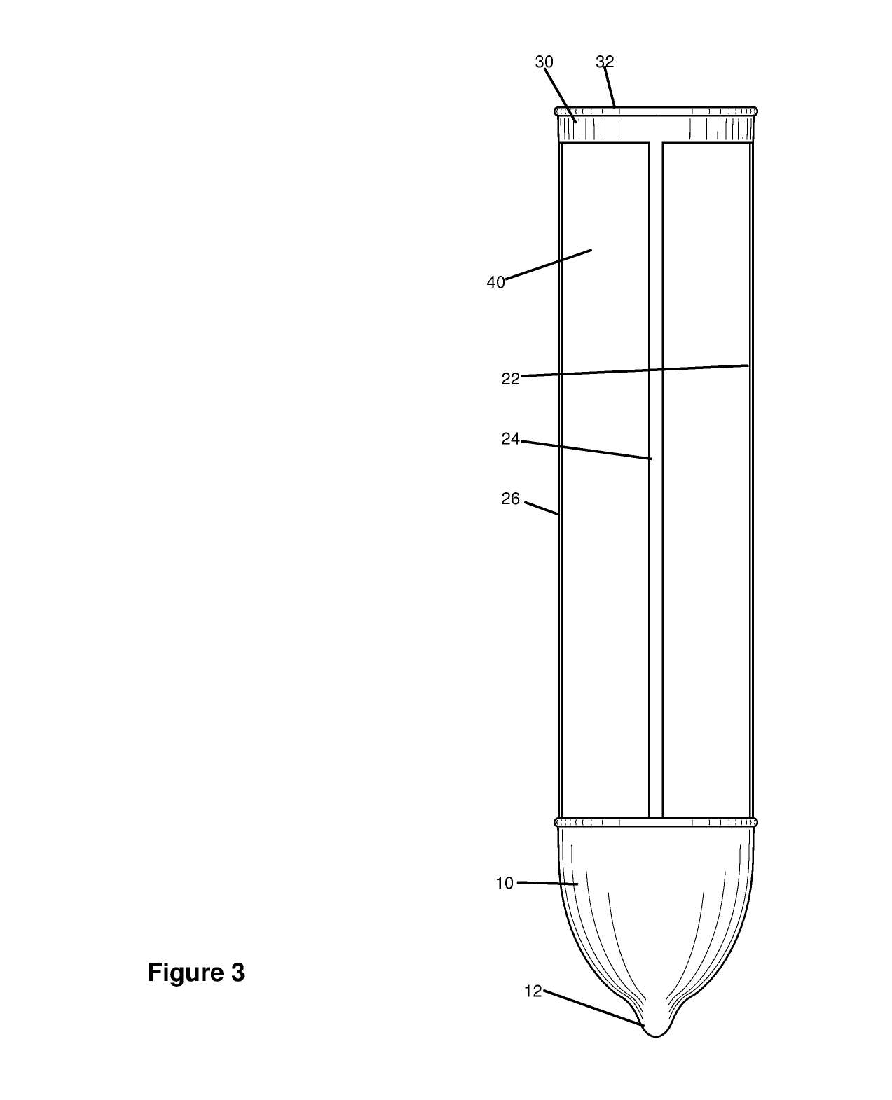

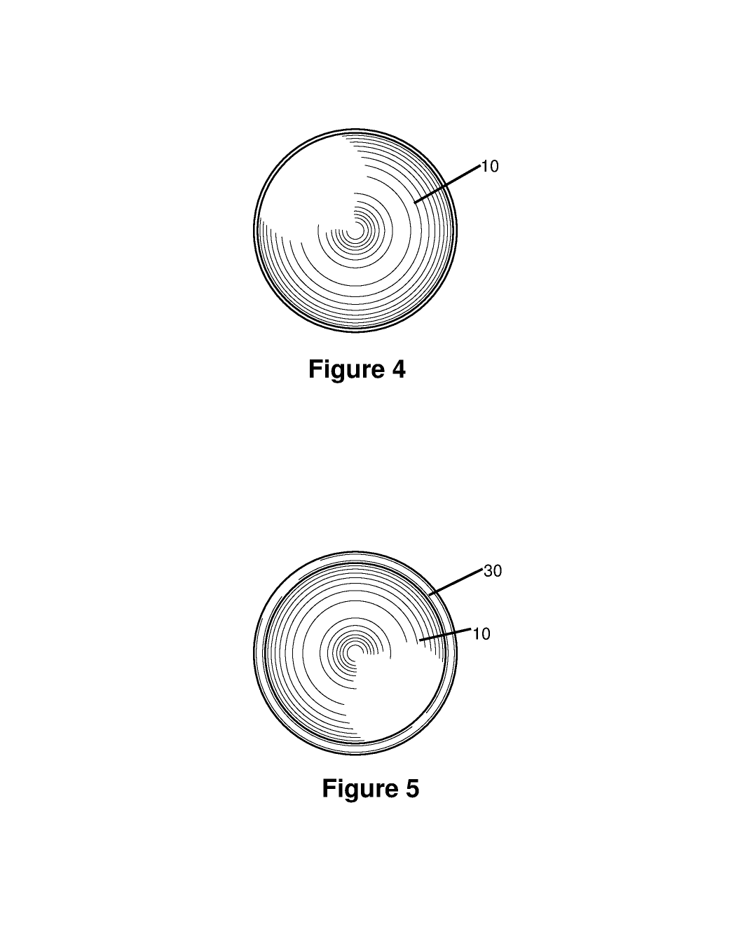

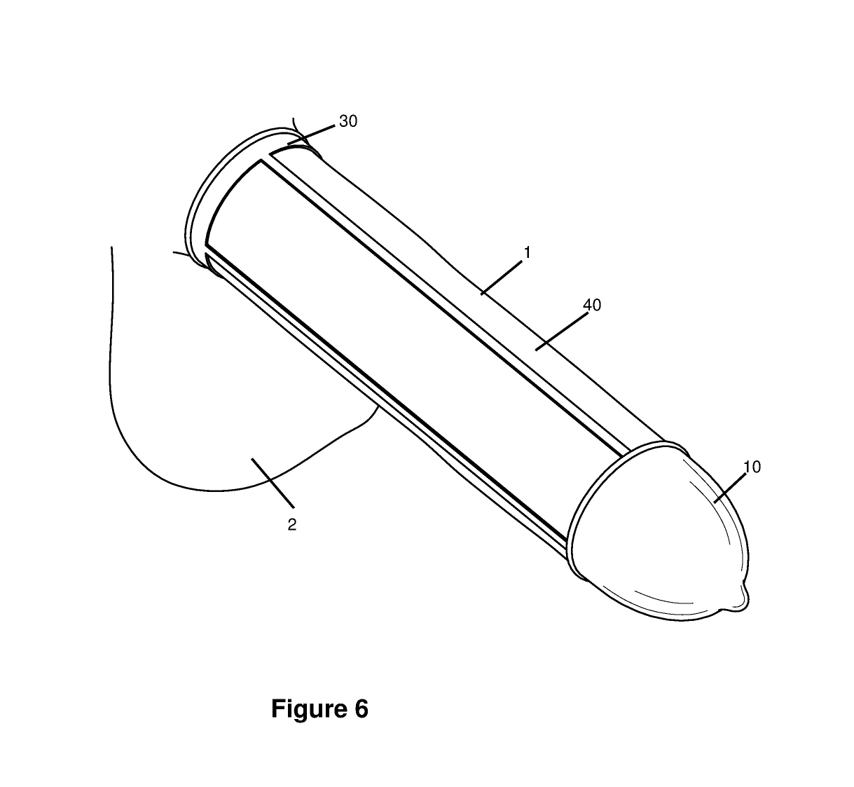

FIGS. 1-5 will be discussed in conjunction with each other. FIG. 1 shows a top perspective view of a portal condom, in an embodiment of the disclosed technology. FIG. 2 shows a bottom perspective view of the portal condom of FIG. 1. FIG. 3 shows a side elevation view of the portal condom of FIG. 1. FIG. 4 shows a top plan view of the portal condom of FIG. 1. FIG. 5 shows a bottom plan view of the portal condom of FIG. 1. The tip 10 of the condom has a reservoir 12 and lowest circumferential ring/largest circumference 14 of the tip. The exterior circumference of the lowest circumferential ring is larger than the interior circumference, and can also be larger on the exterior side, at a portion which is the same interior circumference as other lateral cross-sections thereof. The tip 10 is adapted, configured, designed to, or does cover a tip of a urethra. The tip of a urethra can simply be a portion thereof which it covers extending from an extreme tip where a portal into the penis exists, or can be a widest “mushroom cap” section of the penis, known as the glans penis and/or corona.

Cords are elongated elements which connect a tip 10 to a base ring 30. In the embodiment shown in FIG. 1, there are four equi-sized and equi-spaced cords from one another, cords 22, 24, 26, and 28. A circular base ring 30 with a lip 32 (the lip having a slightly wider exterior circumference) is also shown. The base ring forms a circular portal where the penis tip enters before reaching the tip 10 of the condom. Thus, an interior space 40 is enclosed by the tip 10, cords 22, 24, 26, and 28, and base ring 30. When the penis is inserted into the condom, the portal in the base ring 30 is sealed while the portals (also numbered 40 as the arrow points to the same place—the open space) between the cords allow the penis to be open to the outside and subject to abrasive forces against another object, such as a vaginal wall.

FIG. 4 shows a top plan view of the portal condom of FIG. 1. FIG. 5 shows a bottom plan view of the portal condom of FIG. 1. From the top and bottom view, one can see the interior side of the tip 10. The tip 10 and base 30 remain constant throughout many of the variations of the disclosed technology, where the cord number is changed or diagonals are added.

FIG. 6 shows a perspective view of the portal condom of FIG. 1 with a urethra inserted therein. Here, the urethra 1 fills the interior space 40 of the condom but is exposed through each portal 40 of the condom, the portals being those which are broken by a cord or cords. In some embodiments, the base ring 30 abuts the rest of a person's body 2.

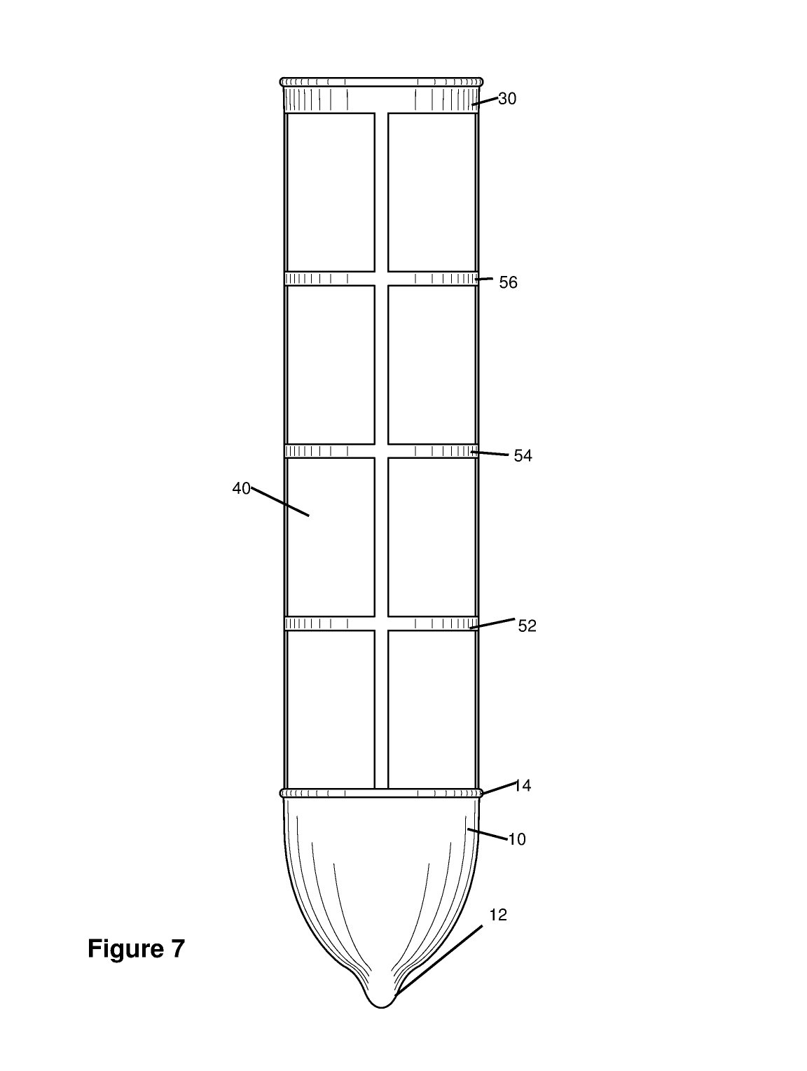

FIG. 7 shows an alternative embodiment of the portal condom with cross-connected rings, in an embodiment of the disclosed technology. The device is as shown with respect to prior figures, except that lateral rings 52, 54 and 56 are shown. Any number of lateral rings can be added. These rings are perpendicular to the cords. For example, lateral ring 54 is perpendicular to cord 22. These lateral rings add stability to the condom, while still allowing the majority of the urethra to be exposed to a world outside of the condom where hopes and dreams can still be achieved. Each lateral ring 54 splits a larger portal 40 into smaller portals.

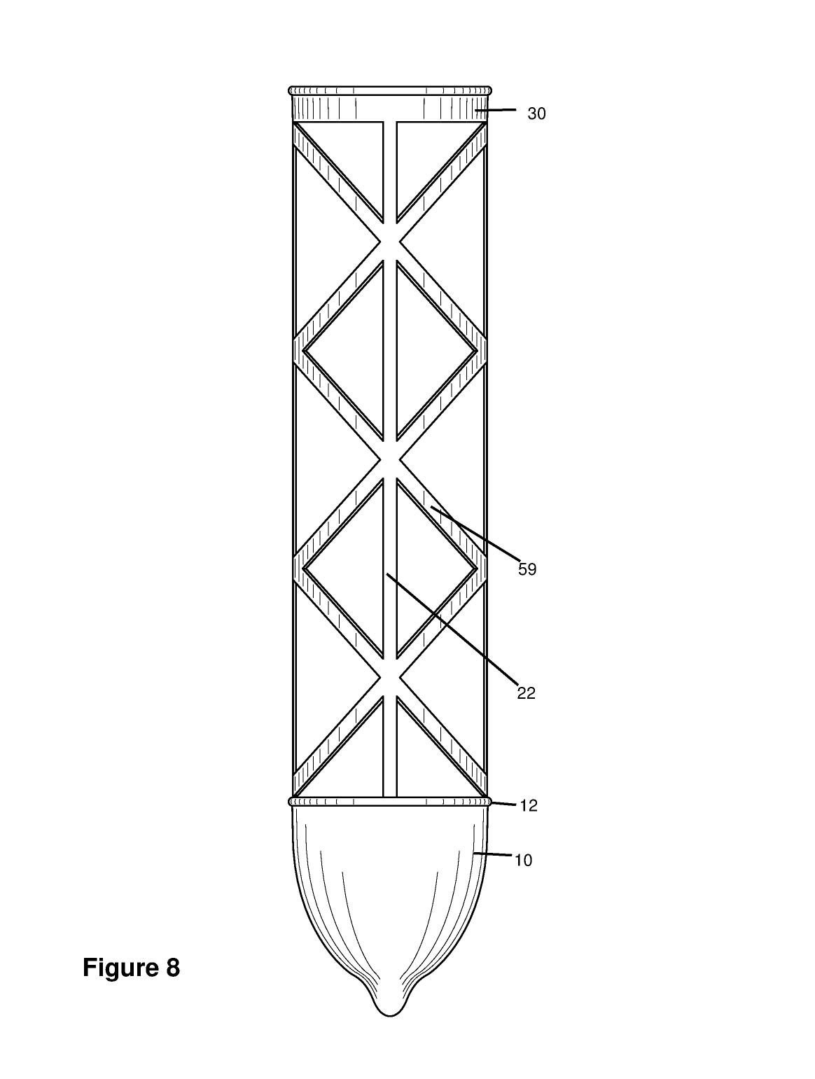

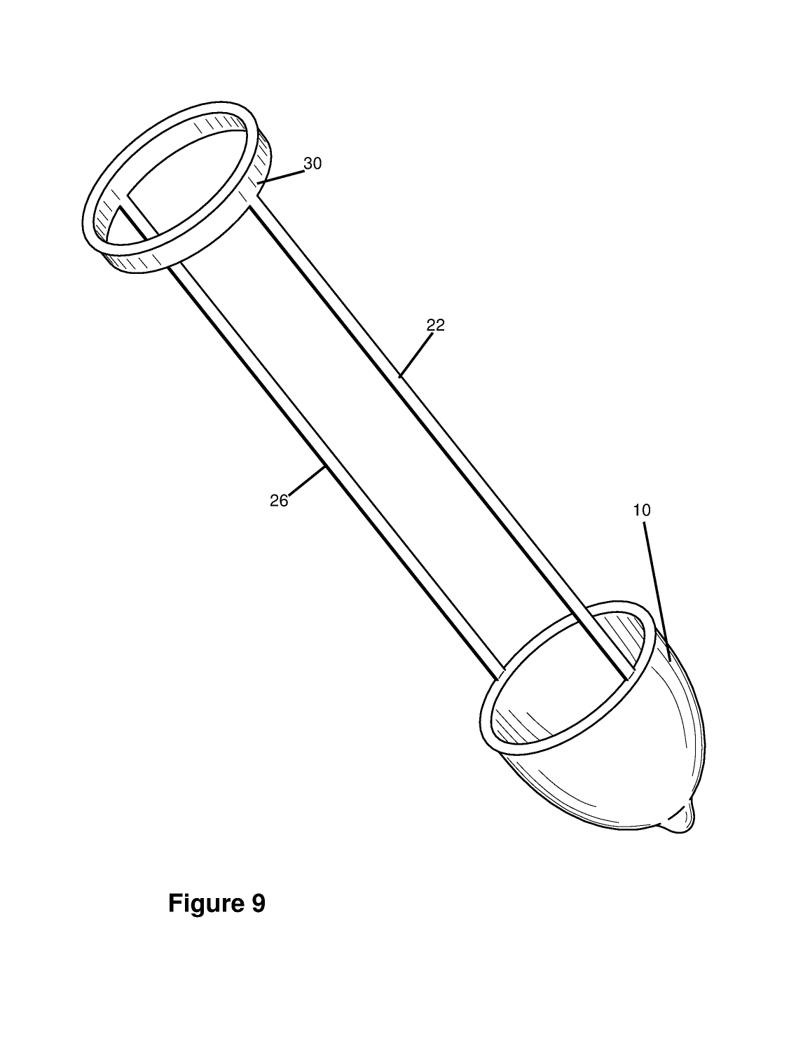

FIG. 8 shows a second alternative embodiment of the portal condom with diamond connected rings, in an embodiment of the disclosed technology. In this embodiment, triangular portals are created as shown. A hypotenuse of each triangle, in some embodiments, is created by a cord such as cord 22, and the legs are created by diagonals 59 which run at 45 degree angles, or another acute angle to one or both of the tip 10 and a cord. This can create a diamond-shaped pattern, as shown in the figure.

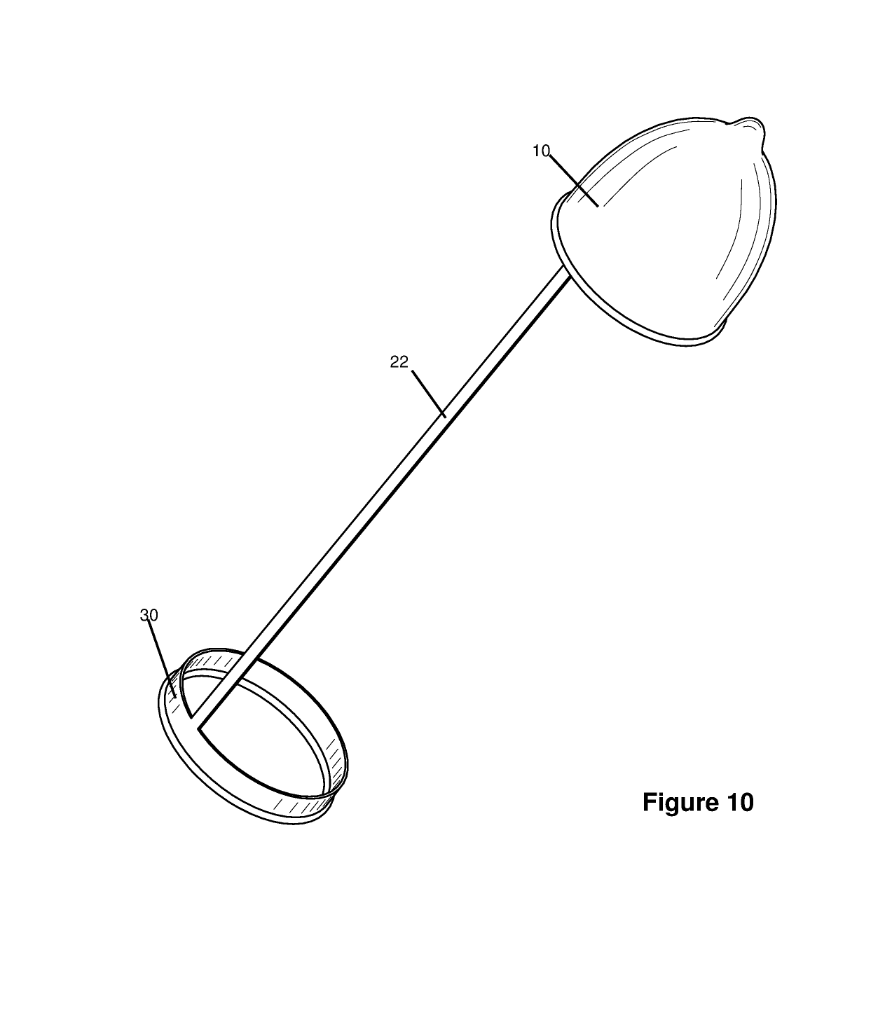

FIG. 9 shows a third alternative embodiment of the portal condom with two cords. FIG. 10 shows a third alternative embodiment of the portal condom with one cords. In these embodiments, the tip 10 and one cord 22, or two cords 22 and 26, are shown connecting the tip 10 to the base 30.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

1. A condom, comprising:
a solid tip;
at least one cord extending from said tip to a base ring;
said base ring extending circumferentially and in parallel to a largest circumference of said solid tip, when said cord extends perpendicularly to said largest circumference of said solid tip and said base ring.

2. The condom of claim 1, wherein said at least one cord is two cords, which are in parallel to each other and connect 180 degrees apart from each other to each of said base ring and said largest circumference of said solid tip.

3. The condom of claim 1, wherein said at least one cord is four cords which are in parallel to each other and connect 90 or 180 degrees apart from each other to each of said base ring and said largest circumference of said solid tip.

4. The condom of claim 3, further comprising a plurality of lateral rings crossing over each of said four cords and being in parallel to said base ring and said largest circumference of said solid tip.

5. The condom of claim 4, wherein said lateral rings are equi-spaced from each other.

6. The condom of claim 2, further comprising a plurality of diagonals extending at a 45 degree angle to said base ring and each said cord.

7. The condom of claim 6, further comprising two sets of said plurality of diagonals each 180 degrees to each other.

8. The condom of claim 7, wherein triangular portals are formed with a hypotenuse along a cord of said at least one cord and each leg is along a diagonal of said plurality of diagonals.

9. The condom of claim 1, wherein at least one portal extends from said tip to said base ring and extends in a space between said base ring and said largest circumference of said tip, except along said at least one cord.

10. The condom of claim 9, wherein said tip is configured to cover a tip of urethra, said base ring being configured to cover a lower portion of said urethra, and an inner cavity is defined by space between said base ring, said at least one cord, and said tip; and
wherein said inner cavity is configured to be filled with a urethra.

11. The condom of claim 2, wherein there are two equi-sized portals, each being said tip, said two cords, and said base ring.

12. The condom of claim 3, wherein there are four equi-sized portals, each being between said tip, two of said four cords, and said base ring.

13. The condom of claim 1, wherein said solid tip, said at least one cord, and said base ring are continuous, uninterrupted, and formed simultaneously to each other.

14. A method of using said condom of claim 1, comprising:
pulling said solid tip over a tip of said urethra;
pulling said base ring over a lower part of a urethra;
straightening said at least one cord until said base ring is parallel to said largest circumference of said tip;
engaging in an activity which causes ejaculation into said tip while said base ring remains substantially in place.
